(19) United States
(12) Patent Application Publication
(10) Pub. No.: US 2022/0289646 A1
(43) Pub. Date: Sep. 15, 2022

(54) METHOD FOR PRODUCING 1,3-BUTADIENE

(71) Applicants: ENEOS MATERIALS CORPORATION, Minato-ku,Tokyo (JP); ENEOS Corporation, Chiyoda-ku (JP)

(72) Inventors: Mayu SUGIMOTO, Minato-ku (JP); Yuichiro SASAKI, Minato-ku (JP)

(73) Assignees: JSR Corporation, Minato-ku (JP); ENEOS Corporation, Chiyoda-ku (JP)

(21) Appl. No.: 17/763,533

(22) PCT Filed: Aug. 26, 2020
(86) PCT No.: PCT/JP2020/032166

§ 371 Date: Mar. 24, 2022

(30) Foreign Application Priority Data
Sep. 26, 2019 (JP) 2019-175043

(51) Int. Cl. C07C 5/48

(52) U.S. Cl. CPC C07C 5/48; C07C 2523/843

(57) ABSTRACT

The present invention has as its object the provision of a method for producing 1,3-butadiene capable of efficiently purifying an absorption solvent while a high productivity is assured.

A method for producing 1,3-butadiene includes: a step (A) of obtaining a produced gas containing 1,3-butadiene; a step (B) of cooling the produced gas; a step (C) of separating the produced gas, which has been subjected to the step (B); a step (D1) of separating the absorption solvent, that has absorbed an absorption component comprising the other gases containing 1,3 -butadiene into an absorption solvent that does not substantially contain the absorption component and an absorption solvent that contains the absorption component; a step (D2) of separating the absorption solvent that contains the absorption component into an absorption solvent that contains a reaction by-product and a 1,3-butadiene liquid; and a step (E) of purifying the absorption solvent, that contains the reaction by-product.

### TECHNICAL FIELD

The present invention relates to a method for producing 1,3-butadiene, and in particular, relates to a method for producing 1,3-butadiene using an oxidative dehydrogenation reaction.

### BACKGROUND ART

Conventionally, as a method for producing 1,3-butadiene (hereinafter also simply referred to as “butadiene”), there has been adopted a method in which components other than butadiene are separated by distillation from a fraction that is obtained by cracking of naphtha and contains molecules with four carbon atoms (hereinafter also referred to as “C4 fraction”).

Demand for butadiene as a raw material for a synthetic rubber or the like has increased, but the amount of C4 fraction supplied has decreased due to a circumstance in which a method for producing ethylene has been changed from a method based on cracking of naphtha to a method based on pyrolysis of ethane. Therefore, production of butadiene in which the C4 fraction is not adopted as a raw material is required.

Regarding methods for producing butadiene, attention has been paid to a method in which butadiene is isolated and obtained from a produced gas that has been obtained by oxidative dehydrogenation of n-butene (for example, see Patent Literatures 1 and 2).

In this production method, n-butene and a molecular oxygen-containing gas containing molecular oxygen (for example, air) are subjected to an oxidative dehydrogenation reaction to obtain a produced gas. The oxidative dehydrogenation reaction is performed under a condition, implemented from the viewpoint of safety, where the concentrations of n-butene and molecular oxygen are adjusted by water (water vapor) and inert gases (for example, molecular nitrogen). The obtained produced gas contains unreacted molecular oxygen and inert gases in addition to butadiene, which is a targeted end product. For this reason, the produced gas is brought into contact with an absorption solvent that contains an organic solvent such as toluene as a main component, so that the absorption solvent selectively absorbs butadiene. As a result, butadiene is separated from the molecular oxygen and the inert gases.

In the process for selectively absorbing butadiene into the absorption solvent, the absorption solvent is required in a large amount and is thus usually circulated and used. The absorption solvent also contains not only butadiene but also reaction by-products of oxidative dehydrogenation, which are also contained in the produced gas. Therefore, separation of butadiene from the absorption solvent, which has been in contact with the produced gas, and removal of the reaction by-products, in other words purification of the absorption solvent that has been in contact with the produced gas, are required for circulation and use of the absorption solvent.

However, a large amount of energy has been necessary because of the purification conventionally requiring a large amount of solvent.

### CITATION LIST

### Patent Literature

Patent Literature 1: Japanese Patent Application Laid-Open No. 2015-189676
Patent Literature 2: International Publication No. 2016/150940

### SUMMARY OF INVENTION

### Technical Problem

The present invention has been made in view of the foregoing circumstances as a result of extensive investigation into a method for producing 1,3-butadiene using an oxidative dehydrogenation reaction by the present inventors. The present invention has as its object the provision of a method for producing 1,3-butadiene capable of efficiently purifying an absorption solvent while assuring a high productivity.

### Solution to Problem

A method for producing 1,3-butadiene of the present invention includes:

a step (A) of performing an oxidative dehydrogenation reaction, with a molecular oxygen-containing gas in the presence of a metal oxide catalyst, of a raw material gas, which contains n-butene, to obtain a produced gas containing 1,3-butadiene;

a step (B) of cooling the produced gas obtained in the step (A);

a step (C) of separating the produced gas, which has been subjected to the step (B), into molecular oxygen and inert gases, and other gases containing 1,3-butadiene by selective absorption into an absorption solvent; and

a step (D) of separating the absorption solvent, which has been obtained in the step (C), that has absorbed the other gases containing 1,3-butadiene, to obtain a 1,3-butadiene liquid, containing 1,3-butadiene, and the absorption solvent, wherein

the step (D) includes:
a step (D1) of separating the absorption solvent, which has absorbed the other gases containing 1,3-butadiene, into an absorption solvent that does not substantially contain an absorption component including the other gases containing 1,3-butadiene and an absorption solvent that contains the absorption component;
a step (D2) of separating the absorption solvent, which has been obtained in the step (D1), that contains the absorption component into an absorption solvent that contains a reaction by-product and the 1,3-butadiene liquid containing 1,3-butadiene; and
a step (E) of purifying the absorption solvent, which has been obtained in the step (D2), that contains the reaction by-product.

In the method for producing 1,3-butadiene of the present invention, it is preferable that the absorption solvent, which has been obtained in the step (D1), that does not substantially contain the absorption component and the purified absorption solvent that has been obtained in the step (E) are returned to the step (C), and

that the concentrations of ketones and aldehydes in the absorption solvent returned from the steps (D1) and (E) to the step (C) are 0% by mass or more and not more than 1% by mass.

In the method for producing 1,3-butadiene of the present invention, it is preferable that in the step (D), the amount of the absorption solvent, which is subjected to the step (D1), that has absorbed the other gases containing 1,3-butadiene is larger than the amount of the absorption solvent, which is subjected to the step (D2), that contains the absorption component.

### Advantageous Effects of Invention

In the method for producing 1,3-butadiene of the present invention, the absorption solvent, which has been obtained in the step (D1), that does not substantially contain the absorption component can be reused as it is without purification. Furthermore, by undergoing the steps (D1), (D2) and (E), absorption solvent, 1,3-butadiene and the reaction by-product are separated from the absorption solvent, which has been obtained in the step (C), that has absorbed the absorption component. This separation can sufficiently suppress mixing of 1,3-butadiene into the absorption solvent containing the reaction by-product. Therefore, energy consumption required for purification of the absorption solvent in the step (E) can be reduced without causing an adverse influence in which the productivity of 1,3-butadiene is reduced due to mixing of 1,3-butadiene into the absorption solvent containing the reaction by-product.

According to the method for producing 1,3-butadiene of the present invention, the absorption solvent can be purified efficiently while a high productivity is assured.

### BRIEF DESCRIPTION OF DRAWINGS

FIG. 1 is a flow diagram illustrating an example of a specific procedure for performing a method for producing 1,3-butadiene of the present invention.

FIG. 2 is a flow diagram illustrating a method for producing 1,3-butadiene according to Comparative Example 1.

### DESCRIPTION OF EMBODIMENTS

Hereinafter, embodiments of the present invention will be described.

A method for producing butadiene (1,3-butadiene) of the present invention has steps shown in (1) to (4) below, and is to produce butadiene (1,3-butadiene) from a raw material gas, which contains n-butene, by performing the steps (1) to (4) described below.

(1) A step (A) of performing an oxidative dehydrogenation reaction, with a molecular oxygen-containing gas in the presence of a metal oxide catalyst, of a raw material gas, which contains n-butene, to obtain a produced gas containing 1,3-butadiene;

(2) A step (B) of cooling the produced gas obtained in the step (A:

(3) A step (C) of separating the produced gas, which has been subjected to the step (B), into molecular oxygen and inert gases, and other gases containing 1,3-butadiene by selective absorption into an absorption solvent;

(4) A step (D) of separating the absorption solvent, which has been obtained in the step (C), that has absorbed the other gases containing 1,3-butadiene, to obtain a 1,3-butadiene liquid, containing 1,3-butadiene, and the absorption solvent.

In the method for producing butadiene of the present invention, the step (D) includes steps (4-1) to (4-3) below:

(4-1) A step (D1) of separating the absorption solvent, which has absorbed the other gases containing 1,3-butadiene, into an absorption solvent that does not substantially contain an absorption component including the other gases containing 1,3-butadiene and an absorption solvent that contains the absorption component;

(4-2) A step (D2) of separating the absorption solvent, which has been obtained in the step (D1), that contains the absorption component into an absorption solvent that contains a reaction by-product and the 1,3 -butadiene liquid containing 1,3-butadiene; and

(4-3) A step (E) of purifying the absorption solvent, which has been obtained in the step (D2), that contains the reaction by-product.

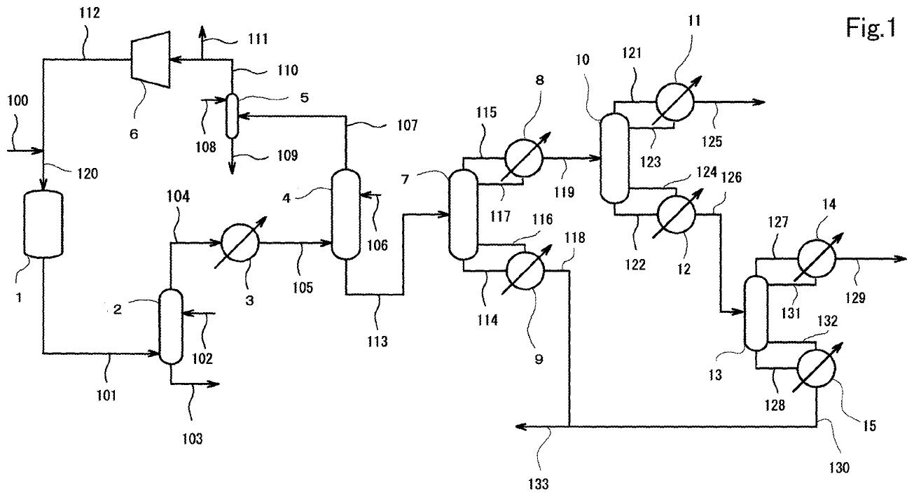

As specific preferable examples of the method for producing butadiene of the present invention, may be mentioned a method in which the absorption solvent obtained in the step (D) (specifically, an absorption solvent, which has been obtained in the step (D1), that does not substantially contain the absorption component and an absorption solvent that has been purified in the step (E)) is returned to the step (C), as illustrated in FIG. 1.

Hereinafter, a specific example of the method for producing butadiene of the present invention will be described in detail using FIG. 1.

FIG. 1 is a flow diagram illustrating an example of a specific procedure for performing the method for producing butadiene of the present invention.

The method for producing butadiene involved in FIG. 1 includes a circulation step of returning the molecular oxygen and the inert gases, which have been obtained in the step (C), to the step (A), or in other words feeding them as a reflux gas to the step (A), in addition to the aforementioned steps (1) to (4).

Step (A):

In the step (A), the raw material gas and the molecular oxygen-containing gas are subjected to an oxidative dehydrogenation reaction in the presence of a metal oxide catalyst to obtain a produced gas containing butadiene (1,3-butadiene). In this step (A), the oxidative dehydrogenation reaction, with the molecular oxygen-containing gas, of the raw material gas is performed in a reactor 1 as illustrated in FIG. 1. Herein, the reactor 1 is a tower-shaped reactor which has a gas inlet provided at an upper portion and a gas outlet provided at a lower portion and includes a catalyst layer (not illustrated in the drawing) formed by filling the inside of the reactor with the metal oxide catalyst. Pipings 100 and 112 are connected to the gas inlet of the reactor 1 via a piping 120, and a piping 101 is connected to the gas outlet.

The step (A) will specifically be described. The raw material gas and the molecular oxygen-containing gas, and, as necessary, inert gases and water (water vapor) are heated to a temperature between about 200° C. and about 400° C. by a preheater (not illustrated), which is disposed between the reactor 1 and the piping 100, and then are supplied to the reactor 1 through the piping 100 that communicates with the piping 120. Together with the raw material gas and the molecular oxygen-containing gas, and inert gases and water (hereinafter also collectively referred to as “newly supplied gas”), which are supplied through the piping 100, the reflux gas from the circulation step, after being heated by the preheater, is supplied to the reactor 1 through the piping 112 that communicates with the piping 120. That is, a mixed gas including the newly supplied gas and the reflux gas is supplied to the reactor 1 after being heated by the preheater. Herein, the newly supplied gas and the reflux gas may each be supplied directly to the reactor 1 through separate pipings. However, it is preferable that the newly supplied gas and the reflux gas in a mixed state are supplied to the reactor 1 through the common piping 120, as illustrated in FIG. 1. When the common piping 120 is provided, a mixed gas that contains various components and is in a state where the components are uniformly mixed in advance can be supplied to the reactor 1. This can prevent a situation where a gas mixed in a nonuniform manner partially forms a detonating gas in the reactor 1.

In the reactor 1 to which the mixed gas has been supplied, butadiene (1,3-butadiene) is produced by an oxidative dehydrogenation reaction, with the molecular oxygen-containing gas, of the raw material gas. Thus, a produced gas containing the butadiene is obtained. The obtained produced gas is discharged from the gas outlet of the reactor 1 to the piping 101.

Raw Material Gas:

The raw material gas is a gaseous substance obtained by gasification, with a vaporizer (not illustrated in the drawing), of n-butene (for example, 1-butene, cis-2-butene and trans-2-butene), which is monoolefin with 4 carbon atoms and is a raw material for 1,3-butadiene. This raw material gas is a combustible gas having combustibility.

The raw material gas may contain optional impurities without impairing the effects of the present invention. As specific examples of the impurities, may be mentioned branched monoolefin such as i-butene and saturated hydrocarbon such as propane, n-butane and i-butane. The raw material gas may contain 1,3-butadiene, which is a targeted end product, as an impurity. The amount of impurities in the raw material gas is usually not more than 60% by volume, and may preferably not more than 40% by volume, more preferably not more than 25% by volume, particularly preferably not more than 1% by volume, per 100% by volume of the raw material gas. When the amount of impurities is too large, there is a tendency for the reaction rate to decrease or the amount of reaction by-product to increase due to a decrease in concentration of n-butene in the raw material gas.

The concentration of n-butene in the raw material gas is usually not less than 40% by volume, and may preferably be not less than 60% by volume, more preferably not less than 75% by volume, particularly preferably not less than 99% by volume.

As the raw material gas, for example, a fraction (raffinate 2) containing n-butene, as a main component, obtained by separating butadiene and i-butene from a C4 fraction (a fraction containing molecules with 4 carbon atoms), which is by-produced by naphtha cracking, or a butene fraction generated by a dehydrogenation reaction or an oxidative dehydrogenation reaction of n-butane may be used. High purity 1-butene, cis-2-butene and trans-2-butene, which are obtained by dimerization of ethylene, and gas mixtures thereof may also be used. In addition, gases containing a large amount of hydrocarbons with 4 carbon atoms (hereinafter, sometimes abbreviated as “FCC-C4”) may be used as raw material gases as they are, and here the gases can be obtained through Fluid Catalytic Cracking by cracking a heavy oil fraction, which is obtained when a crude oil is distilled in a petroleum refining plant or the like, using a powdery solid catalyst in a fluidized bed state to convert the heavy oil fraction into hydrocarbons having low boiling points. Gases obtained by removing impurities such as phosphorus from FCC-C4 may also be used as a raw material gas.

Molecular Oxygen-Containing Gas:

The molecular oxygen-containing gas is usually a gas containing 10 volume % or more of molecular oxygen (O₂). In this molecular oxygen-containing gas, the concentration of molecular oxygen may preferably be not less than 15% by volume, more preferably not less than 20% by volume.

The molecular oxygen-containing gas may include, in addition to molecular oxygen, an optional gas such as molecular nitrogen (N₂), argon (Ar), neon (Ne), helium (He), carbon monoxide (CO), carbon dioxide (CO₂) and water (water vapor). The amount of the optional gas in the molecular oxygen-containing gas is usually not more than 90% by volume, and may preferably be not more than 85% by volume, more preferably not more than 80% by volume when the optional gas is molecular nitrogen, and is usually not more than 10% by volume, and may preferably be not more than 1% by volume when the optional gas is a gas other than molecular nitrogen. When the amount of the optional gas is excessively large, in the reaction system (inside of the reactor 1), molecular oxygen of a required amount may not coexist with the raw material gas. In the step (A), preferred specific examples of the molecular oxygen-containing gas include air.

In the example of the drawing, air is used as the molecular oxygen-containing gas. This molecular oxygen-containing gas that includes air contains at least molecular nitrogen, argon, carbon dioxide and water (water vapor) together with molecular oxygen.

Inert Gases:

It is preferable that inert gases are supplied to the reactor 1 together with the raw material gas and the molecular oxygen-containing gas.

When inert gases are supplied to the reactor 1, the concentrations (relative concentrations) of the raw material gas and the molecular oxygen can be adjusted in such a manner that the mixed gas does not form a detonating gas in the reactor 1.

Examples of the inert gases to be utilized for the method for producing butadiene of the present invention include molecular nitrogen (N₂), argon (Ar) and carbon dioxide (CO₂). These may be used either singly or in any combination of two or more thereof. Among these, molecular nitrogen is preferred from an economic viewpoint.

Water (Water Vapor):

It is preferable that water is supplied to the reactor 1 together with the raw material gas and the molecular oxygen-containing gas.

By supplying water to the reactor 1, it is possible to adjust the concentrations (relative concentrations) of the raw material gas and the molecular oxygen in the same manner as that in the aforementioned inert gases in such a manner that the mixed gas does not form a detonating gas in the reactor 1. Furthermore, coking (deposition of solid carbon) in the metal oxide catalyst can be reduced.

Mixed Gas:

Since the mixed gas contains the combustible raw material gas and the molecular oxygen, the composition thereof is adjusted in such a manner that the concentration of the raw material gas does not fall within an explosive range.

Specifically, the composition of the mixed gas at the gas inlet of the reactor 1 is controlled by monitoring the flow rates with flow meters (not illustrated) installed in the pipings (specifically the piping (not illustrated) communicating with the piping 100, and the piping 112) for supplying respective gases constituting the mixed gas (specifically, the raw material gas, molecular oxygen-containing gas (air), and inert gases and water (water vapor) used as necessary) to the reactor 1.

In this specification, the “explosive range” indicates a range in which the mixed gas has a composition such that it ignites in the presence of some ignition source. Here, it is known that an ignition source that coexists does not ignite a combustible gas when the concentration of the combustible gas is lower than a certain value, and this concentration is referred to as a lower explosion limit. Such a lower explosion limit is the lower limit of the explosive range. It is also known that, when the concentration of the combustible gas is higher than a certain value, an ignition source that coexists does not ignite the combustible gas, and this concentration is referred to as an upper explosion limit. Such an upper explosion limit is the upper limit of the explosive range. These values depend on the concentration of molecular oxygen. In general, the lower the concentration of molecular oxygen is, the more the values approach each other. When the concentration of molecular oxygen becomes a certain value, the values coincide with each other. The concentration of molecular oxygen at this time is referred to as the limit oxygen concentration. Thus, if the concentration of molecular oxygen in the mixed gas is lower than the limit oxygen concentration, the mixed gas does not ignite regardless of the concentration of the raw material gas.

Specifically, in the mixed gas, the concentration of n-butene may preferably be not less than 2% by volume and not more than 30% by volume, more preferably not less than 3% by volume and not more than 25% by volume, particularly preferably not less than 5% by volume and not more than 20% by volume, per 100% by volume of the mixed gas from the viewpoint of productivity of butadiene and suppression of burden on the metal oxide catalyst. If the concentration of n-butene is excessively low, the productivity of butadiene may decrease. On the other hand, when the concentration of n-butene is excessively large, the burden on the metal oxide catalyst may increase.

The concentration (relative concentration) of the molecular oxygen relative to the raw material gas in the mixed gas may preferably be not less than 50 parts by volume and not more than 170 parts by volume, more preferably not less than 70 parts by volume and not more than 160 parts by volume, per 100 parts by volume of the raw material gas. When the concentration of the molecular oxygen in the mixed gas is out of the aforementioned range, there is a tendency in which the concentration of the molecular oxygen at the gas outlet of the reactor 1 is difficult to be adjusted by adjusting the reaction temperature. Since the concentration of the molecular oxygen at the gas outlet of the reactor 1 cannot be controlled by the reaction temperature, decomposition of reaction target product and occurrence of side reaction inside the reactor 1 may not be suppressed.

The concentration (relative concentration) of the molecular nitrogen relative to the raw material gas in the mixed gas may preferably be not less than 400 parts by volume and not more than 1,800 parts by volume, more preferably not less than 500 parts by volume and not more than 1,700 parts by volume, per 100 parts by volume of the raw material gas. The concentration (relative concentration) of water (water vapor) relative to the raw material gas may preferably be 0 part by volume or more and not more than 900 parts by volume, more preferably not less than 80 parts by volume and not more than 300 parts by volume, per 100 parts by volume of the raw material gas. When either the concentration of the molecular nitrogen or the concentration of water is excessively high, the concentration of the raw material gas is decreased with an increase in the concentration of the molecular nitrogen or the concentration of water. Therefore, there is a tendency in which the production efficiency of butadiene decreases. In contrast, when either the concentration of the molecular nitrogen or the concentration of water is excessively low, there is a tendency in which the concentration of the raw material gas falls within an explosive range with a decrease in the concentration of the molecular nitrogen or the concentration of water or removal of heat in a reaction system for adjusting the reaction temperature as described below is difficult.

Metal Oxide Catalyst:

The metal oxide catalyst is not particularly limited as long as the catalyst is capable of functioning as a oxidative dehydrogenation catalyst of the raw material gas, and any known metal oxide catalyst may be used. As such a metal oxide catalyst, for example, those containing an oxide of a metal including at least molybdenum (Mo), bismuth (Bi) and iron (Fe) can be used. Specific preferable examples of such a metal oxide include composite metal oxides represented by the following composition formula (1).

MoₐBi_{b}Fe_{c}X_{d}YₑZᵣO_{g} Composition formula (1):

In the aforementioned composition formula (1), X is at least one selected from the group consisting of Ni and Co. Y is at least one selected from the group consisting of Li, Na, K, Rb, Cs and Tl. Z is at least one selected from the group consisting of Mg, Ca, Ce, Zn, Cr, Sb, As, B, P and W. a, b, c, d, e, f and g each independently show the atomic ratio of each element; when a is 12, b is 0.1 to 8, c is 0.1 to 20, d is 0 to 20, e is 0 to 4, f is 0 to 2, and g is the number of atoms of the oxygen element required to satisfy the atomic valence of each of the aforementioned components.

A composite oxide catalyst containing the composite metal oxide represented by the aforementioned composition formula (1) is highly active and highly selective in a production method of butadiene using an oxidative dehydrogenation reaction, and is further excellent in life stability.

The method for preparing the metal oxide catalyst is not particularly limited, and a known method such as an evaporation and drying method, a spray drying method, or an oxide mixing method using raw materials of respective elements relating to the metal oxide constituting the metal oxide catalyst to be prepared can be adopted.

The raw materials of the aforementioned respective elements are not particularly limited, and examples thereof include an oxide, a nitrate salt, a carbonate salt, an ammonium salt, a hydroxide, a carboxylic acid salt, an ammonium carboxylate salt, an ammonium halide salt, a hydrogenated acid and an alkoxide of the component elements.

Furthermore, the metal oxide catalyst maybe used while being carried on an inert carrier. Examples of the carrier species include silica, alumina and silicon carbide.

Oxygen Dehydrogenation Reaction:

When an oxidative dehydrogenation reaction is initiated in the step (A), it is preferable that supply of the molecular oxygen-containing gas, inert gases and water (water vapor) to the reactor 1 is first initiated, then the amounts supplied of these are adjusted so that the concentration of molecular oxygen at the gas inlet of the reactor 1 is not more than the limit oxygen concentration, and supply of the raw material gas is initiated next, and then the amount supplied of the raw material gas and the amount supplied of the molecular oxygen-containing gas are increased so that the concentration of the raw material gas at the gas inlet of the reactor 1 exceeds the upper explosion limit.

When the amounts supplied of the raw material gas and the molecular oxygen-containing gas are increased, the amount supplied of the mixed gas may be made constant by decreasing the amount supplied of water (water vapor). Thus, the time the gas resides in the pipings (specifically, piping 113) and the reactor 1 can be kept constant, and a change in pressure in the reactor 1 can be suppressed.

The pressure in the reactor 1 (specifically, the pressure at the gas inlet of the reactor 1) that is the pressure in the step (A) may preferably be not less than 0.1 MPaG and not more than 0.4 MPaG, more preferably not less than 0.15 MPaG and not more than 0.35 MPaG, further preferably not less than 0.2 MPaG and not more than 0.3 MPaG.

When the pressure in the step (A) is controlled to fall within the aforementioned range, the reaction efficiency in the oxidative dehydrogenation reaction improves.

When the pressure in the step (A) is excessively low, the reaction efficiency in the oxidative dehydrogenation reaction tends to decrease. On the other hand, when the pressure in the step (A) is excessively high, the yield in the oxidative dehydrogenation reaction tends to decrease.

In the oxidative dehydrogenation reaction, a gas hourly space velocity (GHSV) determined by the following expression (1) may preferably be not less than 500 h⁻¹ and not more than 5,000 h⁻¹, more preferably not less than 800 h⁻¹ and not more than 3,000h⁻¹, further preferably not less than 1,000 h⁻¹and not more than 2,500 h⁻¹.

When GHSV is controlled to fall within the aforementioned range, the reaction efficiency in the oxidative dehydrogenation reaction can improve further.

GHSV[h⁻¹]=gas flow rate at atmospheric pressure [Nm³/h]/catalyst layer volume [m³] Expression (1):

The “catalyst layer volume” in the aforementioned expression (1) represents a volume (apparent volume) of the whole catalyst layer containing pores.

Since an oxidative dehydrogenation reaction is an exothermic reaction, the temperature of a reaction system in the oxidative dehydrogenation reaction increases, and a plurality of kinds of reaction by-products may be produced. As the reaction by-products, unsaturated carbonyl compounds with 3 to 4 carbon atoms such as acrolein, acrylic acid, methacrolein, methacrylic acid, maleic acid, fumaric acid, maleic anhydride and methyl vinyl ketone are produced, and the concentration of the reaction by-products in the produced gas increases, causing various adverse influences. Specifically, the aforementioned unsaturated carbonyl compounds are dissolved in the absorption solvent and the like that are circulated and used in the step (C). Thus, impurities easily accumulate in the absorption solvent and the like, and coking (deposition of solid carbon) on the metal oxide catalyst tends to generate.

As examples of a procedure for controlling the concentration of the unsaturated carbonyl compounds to fall within a certain range in the oxidative dehydrogenation reaction, maybe mentioned a method for adjusting the reaction temperature of the oxidative dehydrogenation reaction. When the reaction temperature is adjusted, the concentration of molecular oxygen at the gas outlet of the reactor 1 can be within a certain range.

Specifically, the reaction temperature may preferably be not lower than 300° C. and not higher than 400° C., more preferably not lower than 320° C. and not higher than 380° C.

When the reaction temperature is controlled to fall within the aforementioned range, coking (deposition of solid carbon) can be suppressed in the metal oxide catalyst, and the concentration of the unsaturated carbonyl compounds in the produced gas can be within a certain range. Furthermore, the concentration of molecular oxygen at the gas outlet of the reactor 1 can also be within a certain range.

On the other hand, when the reaction temperature is excessively low, the conversion rate of n-butene may decrease. When the reaction temperature is excessively high, the concentration of the unsaturated carbonyl compounds increases, and there is a tendency for impurities to accumulate in the absorption solvent and the like, or coking to occur in the metal oxide catalyst.

Herein, as specific preferable examples of the method for adjusting the reaction temperature, may be mentioned a procedure in which the reactor 1 is appropriately cooled by removing heat with a heating medium (specifically, dibenzyltoluene, a nitrite salt or the like), to control the temperature of the catalyst layer to be constant.

Produced Gas:

The produced gas contains a reaction by-product, an unreacted raw material gas, unreacted molecular oxygen, inert gases, water (water vapor) and the like in addition to 1,3-butadiene, which is a reaction target product of the oxidative dehydrogenation reaction, with the molecular oxygen-containing gas, of the raw material gas.

As the reaction by-product, may be mentioned, acetaldehyde, crotonaldehyde, benzaldehyde, acetophenone, benzophenone, fluorenone, anthraquinone, phthalic acid, crotonic acid, tetrahydrophthalic acid, isophthalic acid, terephthalic acid, methacrylic acid, phenol and benzoic acid, in addition to the unsaturated carbonyl compounds with 3 to 4 carbon atoms described above.

The concentration of molecular nitrogen in the produced gas discharged from the reactor 1 may preferably be not less than 35% by volume and not more than 90% by volume, more preferably not less than 45% by volume and not more than 80% by volume. The concentration of water (water vapor) may preferably be not less than 5% by volume and not more than 60% by volume, more preferably not less than 8% by volume and not more than 40% by volume. The concentration of butadiene may preferably be not less than 2% by volume and not more than 15% by volume, more preferably not less than 3% by volume and not more than 10% by volume. The concentration of n-butene may preferably be 0% by volume or more and not more than 2% by volume, more preferably not less than 0.1% by volume and not more than 1.8% by volume.

When the concentration of each component in the produced gas falls within the aforementioned range, the efficiency of butadiene purification, which is performed after the following steps, can be improved, and a side reaction of butadiene that occurs during purification can be suppressed. Thus, energy consumption during production of butadiene can be reduced.

Step (B):

In the step (B), the produced gas obtained in the step (A) is cooled. In this step (B), cooling of the produced gas from the step (A) is usually performed by a quench tower 2 and a heat exchanger for cooling 3, as illustrated in FIG. 1.

The step (B) will specifically be described. The produced gas from the step (A), that is, the produced gas discharged from the reactor 1 is fed to the quench tower 2 through the piping 101, cooled by the quench tower 2, then fed to the heat exchanger for cooling 3 through a piping 104, and further cooled by the heat exchanger for cooling 3. After the step (B) of cooling the produced gas by the quench tower 2 and the heat exchanger for cooling 3 as described above, the produced gas (hereinafter also referred to as “cooled produced gas”) is discharged from the heat exchanger for cooling 3 to a piping 105.

By undergoing this step (B), the produced gas from the step (A) is purified. Specifically, a part of the reaction by-products contained in the produced gas from the step (A) is removed.

Quench Tower:

The quench tower 2 is configured to bring the produced gas from the step (A) into countercurrent contact with a cooling medium to cool the produced gas to a temperature between about 30° C. and about 90° C. In the quench tower 2, a gas inlet for introducing the produced gas from the step (A) is provided at a lower portion, and a medium inlet for introducing the cooling medium is provided at an upper portion. The piping 101 having an end connected to the gas outlet of the reactor 1 is connected to the gas inlet, and a piping 102 is connected to the medium inlet. In the quench tower 2, a gas outlet for discharging the produced gas, from the step (A), that has been cooled by the cooling medium is provided at a tower top, and a medium outlet for discharging the cooling medium which has been in contact (countercurrent contact) with the produced gas from the step (A) is provided at a tower bottom. The piping 104 is connected to the gas outlet, and a piping 103 is connected to the medium outlet.

In the example of this drawing, the cooling medium, which has been in contact (countercurrent contact) with the produced gas from the step (A) and discharged from the quench tower 2, is collected through the piping 103 and appropriately treated, thereby removing the reaction by-products (specifically, organic acids described below). Thus, the cooling medium is reused.

In the quench tower 2, for example, water or an aqueous alkali solution is used as a cooling medium.

The temperature of the cooling medium (temperature thereof at the medium inlet) is appropriately set depending on the intended cooling temperature, and may preferably be not lower than 10° C. and not higher than 90° C., more preferably not lower than 20° C. and not higher than 70° C., particularly preferably not lower than 20° C. and not higher than 40° C.

Furthermore, the temperature inside the quench tower 2 during operation may preferably be not lower than 0° C. and not higher than 100° C., more preferably not lower than 20° C. and not higher than 90° C.

In addition, it is preferable that the pressure in the quench tower 2 during operation (specifically, the pressure at the gas outlet of the quench tower 2), that is, the pressure in the step (B) is equal to or less than the pressure in the step (A).

Specifically, the difference between the pressure in the step (B) and the pressure in the step (A), that is, a value obtained by subtracting the value of the pressure in the step (B) from the value of the pressure in the step (A) may preferably be 0 MPaG or more and not more than 0.05 MPaG, more preferably not less than 0.01 MPaG and not more than 0.04 MPaG.

When the pressure difference between the step (A) and the step (B) is controlled to fall within the aforementioned range, condensation and dissolution into the cooling medium of the reaction by-products in the produced gas from the step (A) can be promoted in the quench tower 2. As a result, the concentration of the reaction by-products in the produced gas, discharged from the quench tower 2, can be further reduced.

The produced gas discharged from the quench tower 2 contains, in addition to butadiene, n-butene, molecular oxygen, inert gases and water (water vapor). The produced gas can also contain reaction by-products (specifically, ketones and aldehydes).

The concentration of molecular nitrogen in the produced gas discharged from the quench tower 2 may preferably be not less than 60% by volume and not more than 94% by volume, more preferably not less than 70% by volume and not more than 90% by volume. The concentration of butadiene may preferably be not less than 2% by volume and not more than 15% by volume, more preferably not less than 3% by volume and not more than 10% by volume. The concentration of water (water vapor) may preferably be not less than 5% by volume and not more than 60% by volume, more preferably not less than 10% by volume and not more than 45% by volume. The concentrations of ketones and aldehydes may preferably be 0% by volume or more and not more than 0.3% by volume, more preferably not less than 0.05% by volume and not more than 0.25% by volume. Ketones and aldehydes contained in the produced gas discharged from the quench tower 2 as the reaction by-product are at least one type of compound selected from the group consisting of methyl vinyl ketone, acetaldehyde, acrolein, methacrolein, crotonaldehyde, benzaldehyde, acetophenone, benzophenone, anthraquinone and fluorene.

When the concentration of each component in the produced gas discharged from the quench tower 2 falls within the aforementioned range, the efficiency of butadiene purification, which is performed after the following steps, can be improved, and a side reaction of butadiene that occurs during purification can be suppressed. Thus, the energy consumption during production of butadiene can be reduced.

The cooling medium, which is discharged from the quench tower 2, that has been in contact with the produced gas from the step (A) may contain the reaction by-product (for example, organic acid) and the like in the produced gas from the step (A) where the reaction by-product has been condensed or dissolved in the cooling medium in the quench tower 2.

The concentration of organic acid in the cooling medium discharged from the quench tower 2 may preferably be 0% by mass or more and not more than 7% by mass, more preferably not less than 1% by mass and not more than 6% by mass. Herein, the organic acid contained as the reaction by-product in the cooling medium discharged from the quench tower 2 is at least one type of compound selected from the group consisting of maleic acid, fumaric acid, acrylic acid, phthalic acid, benzoic acid, crotonic acid, tetrahydrophthalic acid, isophthalic acid, terephthalic acid, methacrylic acid and phenol.

Heat Exchanger for Cooling:

As the heat exchanger for cooling 3, a heat exchanger that is capable of cooling the produced gas, which has been discharged from the quench tower 2, to room temperature (not lower than 10° C. and not higher than 30° C.) is appropriately used.

In the example of this drawing, the heat exchanger for cooling 3 has a gas inlet to which the piping 104, which has an end connected to the gas outlet of the quench tower 2, is connected and a gas outlet to which the piping 105 is connected.

It is preferable that the pressure in the heat exchanger for cooling 3 during operation (specifically, the pressure at the gas outlet of the heat exchanger for cooling 3) is equal to the pressure in the quench tower 2 during operation (the pressure at the gas outlet of the quench tower 2).

The concentration of molecular nitrogen in the cooled produced gas discharged from the heat exchanger for cooling 3, that is, the produced gas from the step (A) may preferably be not less than 60% by volume and not more than 94% by volume, more preferably not less than 70% by volume and not more than 85% by volume. The concentration of butadiene may preferably be not less than 2% by volume and not more than 15% by volume, more preferably not less than 3% by volume and not more than 10% by volume. The concentration of water (water vapor) may preferably be not less than 1% by volume and not more than 30% by volume, more preferably not less than 1% by volume and not more than 3% by volume. The concentration of ketones and aldehydes may preferably be 0% by volume or more and not more than 0.3% by volume, more preferably not less than 0.05% by volume and not more than 0.25% by volume.

Step (C):

In the step (C), the produced gas that has been subjected to the step (B), that is, the cooled produced gas is separated (roughly separated) into molecular oxygen and inert gases, and the other gases containing 1,3-butadiene by selective absorption into the absorption solvent. Herein, the “other gases containing 1,3-butadiene” refer to a gas containing at least butadiene and n-butene (unreacted n-butene). Specifically, the other gases may contain the reaction by-products (specifically, ketones and aldehydes) in addition to butadiene and n-butene.

In this step (C), the separation of the cooled produced gas is performed by an absorption tower 4, as illustrated in FIG. 1. Herein, the absorption tower 4 is a tower in which a gas inlet for introducing the cooled produced gas is provided at a lower portion, a medium inlet for introducing the absorption solvent is provided at an upper portion, a liquid outlet for discharging the absorption solvent, which has absorbed gases (specifically, the other gases containing 1,3-butadiene) (hereinafter also referred to as “gas-absorbing liquid”), is provided at a tower bottom, and a gas outlet for discharging a gas that has not been absorbed by the absorption solvent (specifically, molecular oxygen and inert gases) is provided at a tower top. The piping 105, which has an end connected to the gas outlet of the heat exchanger for cooling 3, is connected to the gas inlet. Furthermore, a piping 106 is connected to the medium inlet, a piping 113 is connected to the liquid outlet, and a piping 107 is connected to the gas outlet.

The step (C) will specifically be described. The cooled produced gas from the step (B), that is, the cooled produced gas discharged from the heat exchanger for cooling 3 is fed to the absorption tower 4 through the piping 105, and in synchronization with this feeding, the absorption solvent is supplied to the absorption tower 4 through the piping 106. Thus, the absorption solvent is brought into countercurrent contact with the cooled produced gas, and so the other gases containing 1,3-butadiene in the cooled produced gas are selectively absorbed by the absorption solvent. As a result, the other gases containing 1,3-butadiene, and the molecular oxygen and the inert gases are roughly separated. While the absorption solvent (the gas-absorbing liquid), which has absorbed the other gases containing 1,3-butadiene, is discharged to the piping 113, the molecular oxygen and the inert gases, which have not been absorbed by the absorption solvent, are discharged to the piping 107.

The temperature inside the absorption tower 4 during operation is not particularly limited. In general, molecular oxygen and inert gases are hardly absorbed by the absorption solvent as the temperature inside the absorption tower 4 increases. On the other hand, the absorption efficiency of hydrocarbons such as butadiene (the other gases containing 1,3-butadiene) into the absorption solvent increases as the temperature inside the absorption tower 4 decreases. Thus, the temperature inside the absorption tower 4 may preferably be not lower than 0° C. and not higher than 60° C., more preferably not lower than 10° C. and not higher than 50° C., inconsideration of the productivity of butadiene.

In addition, it is preferable that the pressure in the absorption tower 4 during operation (specifically, the pressure at the gas outlet of the absorption tower 4), that is, the pressure in the step (C) is equal to or less than the pressure in the step (B).

Specifically, the difference between the pressure in the step (C) and the pressure in the step (B), that is, a value obtained by subtracting the value of the pressure in the step (C) from the value of the pressure in the step (B) may preferably be 0 MPaG or more and not more than 0.05 MPaG, more preferably not less than 0.01 MPaG and not more than 0.04 MPaG.

When the pressure difference between the step (B) and the step (C) is controlled to fall within the aforementioned range, absorption of butadiene (the other gases containing 1,3-butadiene) into the absorption solvent in the absorption tower 4 can be promoted. As a result, the amount of the absorption solvent used can be reduced and energy consumption can be reduced.

Absorption Solvent:

As the absorption solvent, those capable of selectively absorbing the other gases containing 1,3-butadiene are used.

Specifically, examples of the absorption solvent include those containing an organic solvent as a main component. As used herein, “containing an organic solvent as a main component” indicates that the content ratio of the organic solvent in the absorption solvent is not less than 50% by mass.

Examples of the organic solvent constituting the absorption solvent include an aromatic compound such as toluene, xylene and benzene, an amide compound such as dimethylformamide and N-methyl-2-pyrrolidone, a sulfur compound such as dimethyl sulfoxide and sulfolane, a nitrile compound such as acetonitrile and butyronitrile, and a ketone compound such as cyclohexanone and acetophenone.

The amount used (amount supplied) of the absorption solvent is not particularly limited, and may preferably be not less than 10 times by mass and not more than 100 times by mass, more preferably not less than 17 times by mass and not more than 40 times by mass, relative to the flow rate (mass flow rate) of the sum of butadiene and n-butene in the cooled produced gas.

When the amount used of the absorption solvent is controlled to fall within the aforementioned range, the absorption efficiency of the other gases containing 1,3-butadiene can improve.

On the other hand, when the amount used of the absorption solvent is excessively large, the energy consumption, which is used in purification for the absorption solvent to be circulated and used, tends to increase. In addition, when the amount used of the absorption solvent is excessively small, the absorption efficiency of the other gases containing 1,3-butadiene tends to decrease.

The temperature (temperature at the solvent inlet) of the absorption solvent may preferably be not lower than 0° C. and not higher than 60° C., more preferably not lower than 0° C. and not higher than 40° C.

When the temperature of the absorption solvent is controlled to fall within the aforementioned range, the absorption efficiency of the other gases containing 1,3-butadiene can further improve.

Circulation Step:

In the circulation step, the molecular oxygen and the inert gases obtained in the step (C) are appropriately treated, as necessary, and are fed as a reflux gas to the step (A). In this circulation step, the molecular oxygen and the inert gases from the step (C) are treated by a solvent-collecting tower 5 and a compressor 6.

This circulation step will specifically be described. The molecular oxygen and the inert gases from the step (C), that is, the molecular oxygen and the inert gases discharged from the absorption tower 4 are fed to the solvent-collecting tower 5 through the piping 107, subjected to a solvent removal treatment, and then fed to the compressor 6 through a piping 110. As necessary, a pressure adjustment treatment is performed. The molecular oxygen and the inert gases from the step (C) that have been subjected to the solvent removal treatment and the pressure adjustment treatment as described above are discharged from the compressor 6 to the piping 112 toward the reaction tower 1.

In the example of this drawing, while the molecular oxygen and the inert gases discharged from the solvent-collecting tower 5 pass through the piping 110, a part of the molecular oxygen and the inert gases is discarded through a piping 111 that communicates with the piping 110. When the piping 111 for discarding a part of the molecular oxygen and the inert gases discharged from the solvent-collecting tower 5 is thus provided, the amount of the reflux gas to be supplied to the step (A) can be adjusted.

Solvent-Collecting Tower:

The solvent-collecting tower 5 is configured to wash the molecular oxygen and the inert gases from the step (C) with water or a solvent to perform the solvent removal treatment of the molecular oxygen and the inert gases. In the solvent-collecting tower 5, a gas inlet for introducing the molecular oxygen and the inert gases from the step (C) is provided at a central portion, and a water inlet for introducing water or a solvent is provided at an upper portion. The piping 107 having an end connected to the gas outlet of the absorption tower 4 is connected to the gas inlet, and a piping 108 is connected to a water or solvent inlet. In the solvent-collecting tower 5, a gas outlet for discharging the molecular oxygen and the inert gases washed with water or the solvent is provided at a tower top, and a water outlet for discharging the water or solvent used in washing the molecular oxygen and the inert gases from the step (C) is provided at a tower bottom. The piping 110 is connected to the gas outlet, and a piping 109 is connected to a water or solvent outlet.

In this solvent-collecting tower 5, the absorption solvent contained in the molecular oxygen and the inert gases from the step (C) is removed, and the thus removed absorption solvent is discharged to the piping 109 together with the water used for washing, so as to be collected through this piping 109. Furthermore, the molecular oxygen and the inert gases from the step (C), which have been subjected to the solvent removal treatment, are discharged to the piping 110.

In addition, the temperature inside the solvent-collecting tower 5 during operation is not particularly limited, and may preferably be not lower than 0° C. and not higher than 80° C., more preferably not lower than 10° C. and not higher than 60° C.

Compressor:

As the compressor 6, a compressor that is capable of increasing the pressure of the molecular oxygen and the inert gases from the solvent-collecting tower 5, as necessary, and adjusting the pressure to a pressure required in the step (A) is appropriately used.

In the example of this drawing, the compressor 6 has a gas inlet to which the piping 110, which has an end connected to the gas outlet of the solvent-collecting tower 5, is connected and a gas outlet to which the piping 112 is connected.

When the pressure in the step (C) is lower than the pressure in the step (A), pressurization by a pressure difference between the step (C) and the step (A) is performed by this compressor 6 according to the pressure difference.

When the pressurization is performed by this compressor 6, the pressure increase is usually small. Therefore, the electric energy consumption of the compressor is kept small.

In the molecular oxygen and the inert gases discharged from the compressor 6, that is, in the reflux gas, the concentration of molecular nitrogen may preferably be not less than 87% by volume and not more than 97% by volume, more preferably not less than 90% by volume and not more than 95% by volume. Furthermore, the concentration of the molecular oxygen may preferably be not less than 1% by volume and not more than 6% by volume, more preferably not less than 2% by volume and not more than 5% by volume.

Step (D):

In the step (D), the 1,3-butadiene liquid is obtained through steps (D1) and (D2) in this order from the gas-absorbing liquid obtained in the step (C). Also in the step (D1), a reusable absorption solvent is obtained through the steps (D1) and (D2), and a step (E) in this order. Herein, the liquid containing 1,3-butadiene obtained in the step (D) contains at least 1,3-butadiene and n-butane.

That is, in the step (D) including the steps (D1), (D2), and (E), the reusable absorption solvent is first obtained in the step (D1). Then, the 1,3-butadiene liquid is obtained in the step (D2), and the reusable absorption solvent is further obtained in the step (E).

Step (D1):

The absorption solvent is separated from the gas-absorbing liquid obtained in the step (C), and so, in the step (D1), the absorption solvent (hereinafter also referred to as “separated absorption solvent (D1)”) and a gas-absorbing liquid, which absorption components including the other gases containing 1,3-butadiene is concentrated in (hereinafter also referred to as “concentrated gas-absorbing liquid”), are obtained. That is, the gas-absorbing liquid from the step (C) is separated by distillation into the separated absorption solvent (D1) and the concentrated gas-absorbing liquid.

In this step (D1), the separation of the gas-absorbing liquid is performed by a desolvation tower 7, a condenser 8, and a reboiler 9, as illustrated in FIG. 1.

Desolvation Tower:

The desolvation tower 7 is configured so that the gas-absorbing liquid from the step (C) is separated by distillation. In the desolvation tower 7, a liquid inlet for introducing the gas-absorbing liquid from the step (C) is provided at a central portion. A gas outlet for discharging the roughly separated concentrated gas is provided at a tower top, and a liquid outlet for discharging the absorption solvent (D1) is provided at a tower bottom. The piping 113, which has an end connected to the liquid outlet of the absorption tower 4, is connected to the liquid inlet, a piping 115 is connected to the liquid outlet at the tower top, and a piping 114 is connected to the liquid outlet at the tower bottom.

In this desolvation tower 7, the gas absorbing liquid is separated (roughly separated) by distillation into the roughly separated concentrated gas and the absorption solvent (D1). Then, the roughly separated concentrated gas is discharged to the piping 115, and the absorption solvent (D1) is discharged to the piping 114.

The pressure inside the desolvation tower 7 is not particularly limited, and the pressure may preferably be not less than 0.03 MPaG and not more than 1.0 MPaG, more preferably not less than 0.2 MPaG and not more than 0.6 MPaG.

The temperature of the desolvation tower 7 during operation at the tower bottom may preferably be not lower than 80° C. and not higher than 190° C., more preferably not lower than 100° C. and not higher than 180° C.

Condenser:

As the condenser 8, a condenser that is capable of further cooling the roughly separated concentrated gas-absorbing liquid from the desolvation tower 7 is appropriately used.

In the example of this drawing, the condenser 8 has a liquid inlet to which the piping 115, which has an end connected to the outlet at the top of the desolvation tower 7, is connected, and a liquid outlet to which a piping 119 and a piping 117, which is a circulation outlet, are connected. The piping 117 has an end connected to a circulation outlet of the condenser 8 and another end connected to a circulation inlet provided at an upper portion of the desolvation tower 7. The piping 117 is for use in feeding the gas-absorbing liquid toward the desolvation tower 7.

Reboiler:

As the reboiler 9, a reboiler that is capable of heating the absorption solvent (D1) from the desolvation tower 7 is appropriately used.

The absorption solvent (D1) discharged from this reboiler 9 to a piping 118 is supplied again to the absorption tower 4 through a piping 133 and the piping 106 as it is without further purification.

In the example of this drawing, the reboiler 9 has a liquid inlet to which a part of the piping 114, which has an end connected to the liquid outlet of the desolvation tower 7, is connected, and a circulation outlet to which a piping 116 is connected. This piping 116 has an end connected to the circulation outlet of the reboiler 9 and another end connected to a circulation inlet provided at a lower portion of the desolvation tower 7.

The absorption solvent (D1) discharged from the reboiler 9 is substantially free of reaction by-products (specifically, free of ketones and aldehydes). Specifically, in the separated absorption solvent (D1), the concentration of the ketones and aldehydes is 0% by mass or more and not more than 1% by mass, and may preferably be 0% by mass or more and not more than 0.05% by mass.

When the concentration of the ketones and aldehydes in the separated absorption solvent (D1) falls within the aforementioned range, the separated absorption solvent (D1) can be used in the step (C) as it is without further purification.

In this step (D1), it is preferable that the amount of the gas-absorbing liquid to be subjected to the step (D1) is larger than the amount of the concentrated gas-absorbing liquid to be subjected to the step (D2).

Specifically, it is preferable that the ratio of the amount of the concentrated gas-absorbing liquid to be subjected to the step (D2) to the amount of the gas-absorbing liquid to be subjected to the step (D1) is 0.01 to 0.1.

Step (D2):

In the step (D2), the concentrated gas-absorbing liquid obtained in the step (D1) is separated by distillation into a 1,3-butadiene liquid containing 1,3-butadiene and a reaction by-product-containing solvent containing reaction by-products (specifically, the ketones and aldehydes).

In this step (D2), the concentrated gas-absorbing liquid is separated by a desolvation tower 10, a condenser 11, and a reboiler 12, as shown in FIG. 1.

The step (D2) will specifically be described. The concentrated gas-absorbing liquid from the step (D1), that is, the concentrated gas-absorbing liquid discharged from the condenser 8 is fed to the desolvation tower 10 through the piping 119 and separated by distillation. By the separation by distillation in this desolvation tower 10, an absorption solvent containing 1,3-butadiene and an absorption solvent containing the reaction by-products are obtained. The absorption solvent containing 1,3-butadiene discharged from the desolvation tower 10 is fed to the condenser 11 through a piping 121 and cooled. Then, the 1,3-butadiene liquid is discharged from the condenser 11 to a piping 125. Herein, the 1,3-butadiene liquid may contain n-butene together with 1,3-butadiene. On the other hand, the absorption solvent containing the reaction by-products, which is discharged from the desolvation tower 10, is fed to the reboiler 12 through a piping 122 and heated.

Desolvation Tower:

The desolvation tower 10 is configured so that the concentrated gas-absorbing liquid from the step (D1) is separated by distillation. In the desolvation tower 10, a liquid inlet for introducing the concentrated gas-absorbing liquid from the step (D1) is provided at a central portion. A liquid outlet for discharging a gas containing 1,3-butadiene is provided at a tower top, and a liquid outlet for discharging the absorption solvent containing the reaction by-products is provided at a tower bottom. The piping 119, which has an end connected to the liquid outlet of the condenser 8, is connected to the liquid inlet, the piping 121 is connected to the gas outlet at the tower top, and a piping 122 is connected to the liquid outlet at the tower bottom.

In this desolvation tower 10, the gas containing 1,3-butadiene and the absorption solvent containing the reaction by-products, which have been separated from the concentrated gas absorbing liquid, are discharged to the piping 121 and the piping 122, respectively.

The pressure inside the desolvation tower 10 is not particularly limited, and the pressure may preferably be not less than 0.03 MPaG and not more than 1.0 MPaG, more preferably not less than 0.2 MPaG and not more than 0.6 MPaG.

The temperature of the desolvation tower 10 during operation at the tower bottom may preferably be not lower than 80° C. and not higher than 190° C., more preferably not lower than 100° C. and not higher than 180° C.

Condenser:

As the condenser 11, a condenser that is capable of cooling the gas containing 1,3-butadiene from the desolvation tower 10 is appropriately used.

In the example of this drawing, the condenser 11 has a liquid inlet to which the piping 121, which has an end connected to the outlet at the tower top of the desolvation tower 10, is connected, and a liquid outlet to which the piping 125 is connected. The condenser 11 has a circulation outlet. To the circulation outlet, a piping 123 is connected. This piping 123 has an end connected to the circulation outlet of the condenser 11 and another end connected to a circulation inlet provided at an upper portion of the desolvation tower 10. The piping 123 is for use in feeding the 1,3-butadiene liquid toward the desolvation tower 10.

Reboiler:

As the reboiler 12, a reboiler that is capable of heating the absorption solvent containing the reaction by-products from the desolvation tower 10 is appropriately used.

In the example of this drawing, the reboiler 12 has a liquid inlet to which a part of the piping 122, which has an end connected to the liquid outlet at the tower bottom of the desolvation tower 10, is connected. The reboiler 12 also has a circulation outlet to which a piping 124 is connected. The piping 124 has an end connected to the circulation outlet of the reboiler 12 and another end connected to a circulation inlet provided at a lower portion of the desolvation tower 10. The piping 124 is for use in feeding the absorption solvent containing the reaction by-products toward the desolvation tower 10 from the reboiler 12.

It is preferable that the amount of the concentrated gas-absorbing liquid to be subjected to the step (D2) is smaller than the amount of the gas-absorbing liquid to be subjected to the step (D1), as described above.

Step (E):

In the step (E), a reaction by-product-containing liquid obtained in the step (D) is purified.

In this step (E), the purification of the reaction by-product-containing liquid is performed by a solvent-collecting tower 13, a condenser 14, and a reboiler 15, as illustrated in FIG. 1.

The step (E) will specifically be described. The reaction by-product-containing liquid from the step (D) is fed to the solvent-collecting tower 13 through a piping 126 and separated by distillation. By the separation by distillation in this solvent-collecting tower 13, an absorption solvent contained in a trace amount in the reaction by-product-containing liquid is separated from the reaction by-product-containing liquid, to obtain the absorption solvent (hereinafter, also referred to as “absorption solvent (E)”) and the reaction by-product-containing liquid in which the reaction by-products are further concentrated. The absorption solvent (E) discharged from the solvent-collecting tower 13 is fed to the reboiler 15 through a piping 128, heated, and discharged to a piping 130. On the other hand, a concentrated reaction by-product-containing gas discharged from the solvent-collecting tower 13 is fed to the condenser 14 through a piping 127 and cooled, and the reaction by-product liquid is discharged from the condenser 14 to a piping 129.

Solvent-Collecting Tower:

The solvent-collecting tower 13 is configured so that the reaction by-product-containing liquid from the step (D) is separated by distillation. In the solvent-collecting tower 13, a liquid inlet for introducing the reaction by-product-containing liquid from the step (D) is provided at a central portion. An outlet for discharging the concentrated reaction by-product-containing liquid is provided at a tower top, and a liquid outlet for discharging the absorption solvent (E) is provided at a tower bottom. The piping 126 having an end connected to the liquid outlet of the heat exchanger for concentration 12 is connected to the liquid inlet, the piping 127 is connected to the liquid outlet at the tower top, and the piping 128 is connected to the liquid outlet at the tower bottom.

In this solvent-collecting tower 13, the reaction by-product-containing liquid is separated into the concentrated reaction by-product-containing gas and the roughly separated absorption solvent (E). Then, the concentrated reaction by-product-containing gas is discharged to the piping 127, and the roughly separated absorption solvent (E) is discharged to the piping 128.

The pressure inside the solvent-collecting tower 13 is not particularly limited, and the pressure may preferably be not less than 0.03 MPaG and not more than 1.0 MPaG, more preferably not less than 0.2 MPaG and not more than 0.6 MPaG.

The temperature of the solvent-collecting tower 13 during operation at the tower bottom may preferably be not lower than 80° C. and not higher than 190° C., more preferably not lower than 100° C. and not higher than 180° C.

Condenser:

As the condenser 14, a condenser that is capable of cooling the concentrated reaction by-product-containing liquid gas from the solvent-collecting tower 13 is appropriately used.

From such a condenser 14, the reaction by-products are discharged to the piping 129. The reaction by-product liquid discharged to this piping 129 is discarded.

In the example of this drawing, the condenser 14 has a liquid inlet to which the piping 127, which has an end connected to the liquid outlet of the solvent-collecting tower 13, is connected, and a reaction by-product liquid outlet to which the piping 129 is connected. The condenser 14 has a circulation outlet. To the circulation outlet, a piping 131 is connected. This piping 131 has an end connected to the circulation outlet of the condenser 14 and another end connected to a circulation inlet provided at an upper portion of the solvent-collecting tower 13. The piping 131 is for use in feeding the concentrated reaction by-product-containing liquid toward the solvent-collecting tower 13.

Reboiler:

As the reboiler 15, a reboiler that is capable of heating the absorption solvent (E) from the solvent-collecting tower 13 is appropriately used.

The absorption solvent (E) discharged from this reboiler 15 to the piping 130 is supplied again to the absorption tower 4 through the pipings 133 and 106 as it is.

In the example of this drawing, the reboiler 15 has a liquid inlet to which a part of the piping 128, which has an end connected to the liquid outlet of the solvent-collecting tower 13, is connected. The piping 130 is connected to a part of the piping 128 and communicates with the piping 106 via the piping 133. The reboiler 15 has a circulation outlet. To the circulation outlet, a piping 132 is connected. This piping 132 has an end connected to the circulation outlet of the reboiler 15 and another end connected to a circulation inlet provided at a lower portion of the solvent-collecting tower 13. The piping 132 is for use in feeding the absorption solvent (E) of the reboiler 15 toward the solvent-collecting tower 13.

The absorption solvent (E) discharged from the reboiler 15 is returned to the step (C) together with the absorption solvent (D1) discharged from the reboiler 9. That is, the absorption solvent (E) discharged from the reboiler 15 to the piping 130 and the absorption solvent (D1) discharged from the reboiler 9 to the piping 118 are mixed in the piping 113, and the mixed absorption solvents are supplied again to the absorption tower 4 through the piping 106.

In the absorption solvent which is supplied again through the piping 133 and the piping 106 in this manner, it is preferable that the concentration of the ketones and aldehydes is 0% by mass or more and not more than 1% by mass.

In the method for producing 1,3-butadiene of the present invention, the separated absorption solvent (D1) is first separated in the step (D1) from the gas-absorbing liquid obtained in the step (C). The 1,3-butadiene liquid is then separated in the step (D2), and the reaction by-product-containing liquid is purified in the step (E) to obtain the separated absorption solvent (E), as described above.

The separated absorption solvent (D1) obtained in the step (D1) can be reused as it is. Therefore, the separated absorption solvent (D1) can be returned to the step (C) as it is without purification. Further, the amount of the reaction by-product-containing liquid which is to be subjected to the step (E) is small. Furthermore, separation of the gas-absorbing liquid into the 1,3-butadiene liquid, the separated absorption solvent (for example, the separated absorption solvent (D1) and the separated absorption solvent (E)) and the reaction by-product liquid is performed by successively undergoing the steps (D1), (D2) and (E). Therefore, mixing of 1,3-butadiene into the reaction by-product liquid, or in other words occurrence of loss of butadiene can be suppressed. Accordingly, energy consumption required for purification of the absorption solvent in the step (E) can be reduced without causing an adverse influence in which the productivity of 1,3-butadiene is reduced due to occurrence of loss of butadiene.

According to the method for producing 1,3-butadiene of the present invention, purification, required for circulation and use, of the absorption solvent can be performed efficiently while a high productivity is assured.

### EXAMPLES

Hereinafter, specific Examples of the present invention will be described. However, the present invention is not limited to these Examples.

A method for analyzing a composition of gas and a method for analyzing ketones and aldehydes are as follows.

Gas composition analysis was performed by gas chromatography under conditions shown in Table 1 below. With respect to water (water vapor (H₂O)), calculation was performed by adding the moisture content obtained by water-cooled trapping during gas sampling.

TABLE 1
SUMMARY OF GAS COMPOSITION ANALYSIS

| | | |
|---|---|---|
| GAS TYPE | 1,3-BUTADIENE, n-BUTENE | N₂, O₂, C0x, H₂O |
| TYPE | GC-14B (MANUFACTURED BY | GC-14B (MANUFACTURED BY |
| | SHIMADZU CORPORATION) | SHIMADZU CORPORATION) |
| DETECTOR | FID | TCD |
| COLUMN | TC-BOND Alumina/Na₂SO₄ | WG-100 |
| | 0.53 mm I.D. × 30 m df = 10 μm | 6. 35 mm I.D. × 1. 8 m |
| | (MANUFACTURED BY | (MANUFACTURED BY |
| | GL SCIENCES INC.) | GL SCIENCES INC.) |
| CARRIER GAS | N₂ 40 ml/min | He 50 ml/min |
| TEMPERATURE | 200 ° C. | 60° C. |
| INJECTION | 250° C. | 80° C. |
| DETECTOR | 60° C. 5 min→135° C.(5° C./ | 50° C. |
| COLUMN | min) →185° C.(15° C./min) | |

Analysis of ketones and aldehydes was performed by liquid chromatography under conditions shown in Table 2 below.

TABLE 2

| | |
|---|---|
| TYPE | LC-2000Plus |
| | (MANUFACTURED BY JASCO |
| | CORPORATION) |
| DETECTOR | UV (210 nm, 230 nm) |
| COLUMN | TSKgel ODS-100 V 5 μm |
| | 4.6 mm ID × 15 cm |
| | (MANUFACTURED BY TOSOH |
| | CORPORATION) |
| ELUENT | ACETONITRILE/PHOSPHORIC |
| | ACID LIQUID 0.8 ml/min |
| COLUMN OVEN | 40° C. |

### Example 1

In accordance with the flow diagram of FIG. 1, 1,3-butadiene was produced from a raw material gas containing n-butene through the following steps (A), (B), (C), (D1), (D2), and (E), and circulation step.

Step (A):

To the reactor 1 (inner diameter: 21.2 mm, outer diameter: 25.4 mm) filled with a metal oxide catalyst so that the length of a catalyst layer was 4,000 mm, a mixed gas with a volume ratio (n-butene/O₂/N₂/H₂O) of 1/1.5/16.3/1.2 was supplied at a gas hourly space velocity (specifically, SV calculated using a flow rate in a standard state) of 2,000 h⁻¹. The raw material gas and a molecular oxygen-containing gas were subjected to an oxidative dehydrogenation reaction under a condition of a reaction temperature of 320 to 330° C., to obtain a produced gas containing 1,3-butadiene. The pressure in this step (A), that was, the pressure at the gas inlet of the reactor 1 was 0.1 MPaG.

In this step (A), as the metal oxide catalyst, a metal oxide catalyst in which an oxide represented by a composition formula: Mo₁₂Bi₅Fe_{0.5}Ni₂Co₃K_{0.1}Cs_{0.1}Sb_{0.2} was carried on spherical silica at a proportion of 20% of the whole catalyst volume was used.

As the mixed gas, the raw material gas and a reflux gas (molecular oxygen and inert gases) were mixed, and as necessary, air as the molecular oxygen-containing gas, molecular nitrogen as the inert gases, and water (water vapor) were further mixed, to adjust a composition.

Step (B):

The produced gas discharged from the reactor 1 was brought into countercurrent contact with water as a cooling medium in the quench tower 2 to be quenched. After being cooled to 76° C., the produced gas was cooled to 30° C. in the heat exchanger 3.

Step (C):

The produced gas discharged from the heat exchanger for cooling 3 (cooled produced gas) was supplied from the gas inlet at the lower portion of the absorption tower 4 (outer diameter: 152.4 mm, height: 7,800 mm, material: SUS304) inside of which a regular packing was disposed, and an absorption solvent containing toluene in an amount of not less than 95% by mass was supplied at 10° C. from the solvent inlet at the upper portion of the absorption tower 4. The amount of the absorption solvent supplied was 33 times by mass the flow rate (mass flow rate) of the sum of butadiene and n-butene in the cooled produced gas. The pressure in this step (C), that was, the pressure at the gas outlet of the absorption tower 4 was 0.1 MPaG.

Circulation Step:

The gas discharged from the absorption tower 4 was washed with water or a solvent in the solvent-collecting tower 5, to remove a small amount of absorption solvent contained in the gas. The gas in which the absorption solvent was thus removed was discharged from the solvent-collecting tower 5, a part of the gas was discarded, and most of the rest was fed to the compressor 6. In the compressor 6, the gas from the solvent-collecting tower 5 was pressurized by a pressure adjustment treatment. The absorption solvent was thus removed, and the pressurized gas was discharged from the compressor 6 and returned to the reaction tower 1.

Step (D): Step (D1):

The liquid discharged from the absorption tower 4 was separated by distillation in the desolvation tower 7, and the separated gas discharged from the outlet at the tower top of the desolvation tower 7 was cooled by the condenser 8, to obtain a concentrated liquid (hereinafter also referred to as “concentrated separated liquid (D1)”). On the other hand, the absorption solvent (hereinafter also referred to as “circulating absorption solvent (D1)”) was obtained at the liquid outlet at the tower bottom of the desolvation tower 7.

Step (D): Step (D2):

The concentrated separated liquid (D1) discharged from the condenser 8 was separated by distillation in a separation tower 10, and the separated gas discharged from the outlet at the tower top of the desolvation tower 10 was cooled by the condenser 11, to obtain a 1,3-butadiene liquid containing 1,3-butadiene. This 1,3-butadiene liquid was collected as a targeted end product. On the other hand, a concentrated liquid containing the reaction by-products (hereinafter also referred to as “concentrated separated liquid (D2)”) was obtained from the liquid outlet at the tower bottom of the desolvation tower 10.

The amount of the concentrated separated liquid (D1) which was subjected to this step (D2) (referred to as (the amount of solvent in the step (D2)) in Table 3) and the amount of the liquid, which was discharged from the absorption tower 4, that was subjected to the step (D1) (referred to as “the amount of solvent in the step (D1)” in Table 3) were confirmed. The amount of the concentrated separated liquid (D1) subjected to the step (D2) was 0.04 times the amount of the liquid subjected to the step (D1).

Step (D): Step (E):

The concentrated separated liquid (D2) discharged from the liquid outlet at the tower bottom of the desolvation tower 10 was separated and purified in the solvent-collecting tower 13, and the absorption solvent (hereinafter also referred to as “circulating absorption solvent (E)”) was obtained from the liquid outlet at the tower bottom of the solvent-collecting tower 13. On the other hand, the separated gas discharged from the outlet at the tower top of the solvent-collecting tower 13 was cooled by the condenser 14, to obtain a reaction by-product liquid containing the reaction by-products. The reaction by-product liquid was discarded.

The circulating absorption solvent (E) obtained in this step (E) was supplied to the absorption tower 4 through the pipings 133 and 106 together with the circulating absorption solvent (D1) obtained in the step (D1). The concentrations of ketones and aldehydes in the mixed liquid of the circulating absorption solvent (D1) and the circulating absorption solvent (E) which passed through the piping 106 (referred to as “circulating absorption solvent” in Table 3) were confirmed to be 300 (wtppm) (0.03% by mass).

The calorie used in the step (E), specifically steam unit consumption in the reboiler 15 was confirmed to be 42 (kcal/raw material butene).

Comparative Example 1

The same procedures as those of Example 1 were performed to produce 1,3-budadiene from the raw material gas containing n-butene, except that in the method for producing 1,3-butadiene according to Example 1, a desolvation tower 21 having liquid outlets at a tower top, a tower bottom and a central part was used instead of the desolvation tower 7 having two outlets provided at the tower top and the tower bottom, the separation liquid discharged from the liquid outlet at the central part of the desolvation tower 21 was supplied to the solvent-collecting tower 13, and the concentrated liquid discharged from the condenser 8 was collected as it is as a targeted end product.

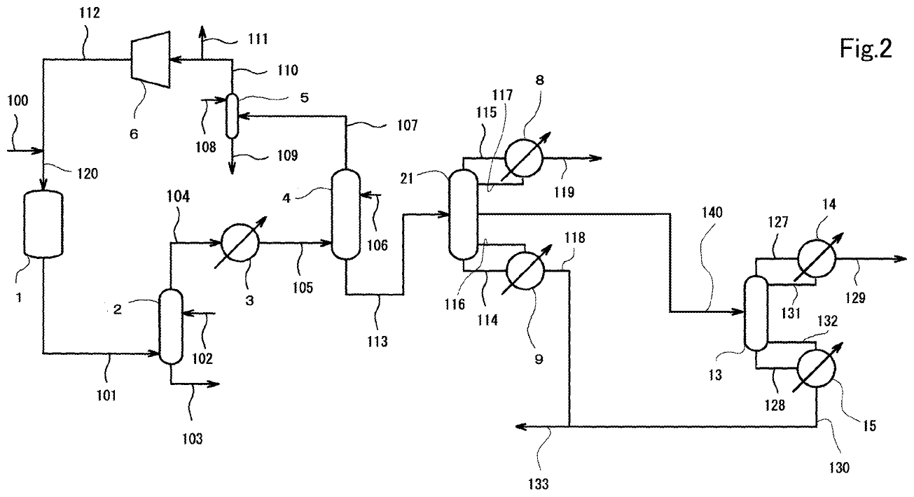

Comparative Example 1 will specifically be described. In a method for producing 1,3-butadiene according to Comparative Example 1, the steps (A), (B) and (C) and the circulation step were performed in the same manner as in the method for producing 1,3-butadiene according to Example 1 in accordance with the flow diagram of FIG. 2. Thus, a liquid discharged from the absorption tower 4 was obtained, and a gas discharged from the absorption tower 4 was returned to the reactor 1. A liquid (liquid from the step (C)) discharged from the absorption tower 4 was separated in the desolvation tower 21 by distillation, and the separated liquid discharged from the liquid outlet at the tower bottom of the desolvation tower 21 was obtained in the same manner as in the step (D1) of the method for producing 1,3-butadiene according to Example 1. A separated liquid discharged from the outlet at the tower top of the desolvation tower 21 was cooled in the condenser 8, and the obtained 1,3-butadiene liquid was collected as a targeted end product. A separated liquid, which was discharged from the liquid outlet at the central part of the desolvation tower 21, was fed to the solvent-collecting tower 13 through the piping 140, and treated in the solvent-collecting tower 13 in the same manner as in the step (E) of the method for producing 1,3-butadiene according to Example 1. As a result, a reaction by-product liquid containing the absorption solvent and the reaction by-product was obtained. In the same manner as in the method for producing 1,3-butadiene according to Example 1, the concentrations of ketones and aldehydes in a mixed liquid of the absorption solvent discharged from the desolvation tower 21 and the absorption solvent discharged from the solvent-collecting tower 13, which were passing through the piping 106, (referred to as “circulating absorption solvent” in Table 3) were confirmed to be 300 wtppm (0.030% by mass).

Steam unit consumption (use calorie) in the reboiler 15 was confirmed to be 65 (kcal/raw material butene).

TABLE 3

| | | COMPAR- |
|---|---|---|
| | | ATIVE |
| | EXAM- | EXAM- |
| | PLE 1 | PLE 1 |
| COMPOSITION OF MIXED GAS: | 1/1.5/16.3/1.2 | |
| n-BUTENE/O₂/N₂/H₂O | | |
| S V [h⁻¹] | 2000 | 2000 |
| PRESSURE IN STEP (A) [MP a G] | 0.1 | 0.1 |
| PRESSURE IN STEP (C) [MP a G] | 0.1 | 0.1 |
| RATIO (BY MASS) OF AMOUNT OF | 33 | 33 |
| ABSORPTION SOLVENT SUPPLIED | | |
| TO TOTAL FLOW RATE (MASS | | |
| FLOW RATE) OF BUTADIENE AND | | |
| n-BUTENE IN COOLED PRODUCED GAS | | |
| PRESENCE OR ABSENCE OF STEP (D2) | PRESENCE | ABSENCE |
| RATIO OF AMOUNT OF SOLVENT IN | 0.04 | — |
| STEP (D2) TO AMOUNT OF SOLVENT | | |
| IN STEP (D1) | | |
| STEAM UNIT CONSUMPTION IN STEP | 40 | 85 |
| (E) (kcal/RAW MATERIAL BUTENE) | | |
| RATIO (BY MASS) OF AMOUNT OF LOSS | 0.001 | 0.003 |
| OF BUTADIENE IN STEP (E) TO | | |
| AMOUNT OF RAW MATERIAL BUTENE | | |
| CONCENTRATIONS OF KETONES AND | 300 | 300 |
| ALDEHYDES IN CIRCULATING | | |
| ABSORPTION SOLVENT (wtppm) | | |

From the results of Table 3, it was confirmed that the energy consumption required for purification of the absorption solvent in the step (E) was reduced by the method for producing 1,3-butadiene according to Example 1.

Furthermore, in the method for producing 1,3-butadiene of the present according to Example 1, it was confirmed that the concentrations of ketones and aldehydes in the absorption solvent returned from the steps (D1) and (E) to the step (C) (circulating absorption solvent) were not more than 1% by mass, and that the amount of the absorption solvent, which was subjected to the step (D), that absorbed the other gases containing 1,3-butadiene was larger than the amount of the absorption solvent, which was subjected to the step (D2), that contained the absorption component.

### REFERENCE SIGNS LIST

1 reactor
2 quench tower
3 heat exchanger for cooling
4 absorption tower
5 solvent-collecting tower
6 compressor
7 desolvation tower
8 condenser
9 reboiler
10 desolvation tower
11 condenser
12 reboiler
13 solvent-collecting tower
14 condenser
15 reboiler
21 desolvation tower
100 to 132, 140 piping

1. A method for producing 1,3-butadiene comprising:
a step (A) of performing an oxidative dehydrogenation reaction, with a molecular oxygen-containing gas in a presence of a metal oxide catalyst, of a raw material gas, which contains n-butene, to obtain a produced gas containing 1,3-butadiene;
a step (B) of cooling the produced gas obtained in the step (A);
a step (C) of separating the produced gas, which has been subjected to the step (B), into molecular oxygen and inert gases, and other gases containing 1,3-butadiene by selective absorption into an absorption solvent; and
a step (D) of separating the absorption solvent, which has been obtained in the step (C), that has absorbed the other gases containing 1,3-butadiene, to obtain a 1,3-butadiene liquid, containing 1,3-butadiene, and the absorption solvent, wherein
the step (D) includes:
a step (D1) of separating the absorption solvent, which has absorbed the other gases containing 1,3-butadiene, into an absorption solvent that does not substantially contain an absorption component including the other gases containing 1,3-butadiene and an absorption solvent that contains the absorption component;
a step (D2) of separating the absorption solvent, which has been obtained in the step (D1), that contains the absorption component into an absorption solvent that contains a reaction by-product and the 1,3-butadiene liquid containing 1,3-butadiene; and
a step (E) of purifying the absorption solvent, which has been obtained in the step (D2), that contains the reaction by-product.

2. The method for producing 1,3-butadiene according to claim 1, wherein:
the absorption solvent, which has been obtained in the step (D1), that does not substantially contain the absorption component and the purified absorption solvent that has been obtained in the step (E) are returned to the step (C), and
concentrations of ketones and aldehydes in the absorption solvent returned from the steps (D1) and (E) to the step (C) are 0% by mass or more and not more than 1% by mass.

3. The method for producing 1,3-butadiene according to claim 1, wherein in the step (D), an amount of the absorption solvent, which is subjected to the step (D1), that has absorbed the other gases containing 1,3-butadiene is larger than an amount of the absorption solvent, which is subjected to the step (D2), that contains the absorption component.

4. The method for producing 1,3-butadiene according to claim 2, wherein in the step (D), an amount of the absorption solvent, which is subjected to the step (D1), that has absorbed the other gases containing 1,3-butadiene is larger than an amount of the absorption solvent, which is subjected to the step (D2), that contains the absorption component.
